(19) United States
(12) Patent Application Publication
(10) Pub. No.: US 2026/0041583 A1
(43) Pub. Date: Feb. 12, 2026

(54) SYSTEM AND METHOD FOR CIRCULATING WATER TO PROVIDE ALTERNATING THERMO- AND CRYO-THERAPY

(71) Applicant: HYPERICE IP SUBCO, LLC, Irvine, CA (US)

(72) Inventors: Alexander Joseph Aguiar, San Diego, CA (US); Trevor Austin Kerth, San Diego, CA (US); Robert Glen Edwards, San Diego, CA (US)

(21) Appl. No.: 19/295,225

(22) Filed: Aug. 8, 2025

Related U.S. Application Data
(60) Provisional application No. 63/680,900, filed on Aug. 8, 2024.

(51) Int. Cl. A61F 7/00; A61F 7/02; A61H 9/00

(52) U.S. Cl. CPC A61F 7/0085; A61H 9/0078; A61F 2007/0056; A61F 2007/0086; A61F 2007/0093; A61F 2007/0244; A61H 2201/0207; A61H 2201/0214; A61H 2201/0264; A61H 2201/5005

(57) ABSTRACT

An apparatus for providing contrast and compression therapy includes a hot water tank, a cold water tank, a water pump, a heat exchanger, and a therapy pad, where the hot and cold water tanks are configured to store water at respective target temperatures for alternating delivery to the therapy pad for alternating thermotherapy and cryotherapy. The apparatus further includes a programmable control unit configured to automatically switch the alternating delivery of the stored water at the respective target temperatures to the therapy pad, and an air pump configured to deliver pressurized air to the therapy pad to provide compression therapy simultaneously with the thermotherapy or the cryotherapy.

### CROSS-REFERENCE TO RELATED APPLICATION

This application claims the benefit of and priority to U.S. Provisional Patent Application No. 63/680,900 filed on Aug. 8, 2024, the entire disclosure of which is hereby incorporated by reference in its entirety for all purposes.

### TECHNICAL FIELD

The application is directed to the field of therapeutic devices, and, more particularly, is directed to the field of devices that provide alternating thermo- and cryo-therapy to selected portions of a body using circulating water.

### BACKGROUND

Thermotherapy is the application of any substance to the body that increases tissue temperature. This results in improved blood flow, tissue metabolism, and connective tissue extensibility. Increased blood flow facilitates tissue healing by supplying protein, nutrients, and oxygen at the site of injury. This increase in metabolism helps the healing process by increasing both the catabolic and anabolic reactions needed to degrade and remove the metabolic by-products of tissue damage. Cryotherapy is the local or general use of low temperatures in medical therapy, or the removal of heat from a body part to relieve pain. The cold causes vasoconstriction, which results in decreased tissue blood flow, and reduces tissue metabolism, oxygen utilization, inflammation, and muscle spasm. While thermotherapy and cryotherapy each find treatment effects towards respective applications, sometimes, neither thermotherapy nor cryotherapy will be sufficient, not on their own. More recent studies indicate that a combination of cold and heat therapy can show improved treatments when compared to thermotherapy or cryotherapy alone. For example, alternating thermo- and cryo-therapy (also referred to as “contrast therapy”) has been shown to exhibit benefits in circulation stimulation, inflammation reduction, and loosened muscles and joints, resulting in improved exercise treatment.

In applying contrast therapy, current options are very limited and many applications require manually changing between thermotherapy and cryotherapy apparatuses, which are costly and not convenient to operate and maintain, not to mention the portability of these apparatuses. In addition, some contrast therapy apparatuses require continuous heat, cold, or water supply and/or replacement from external resources. Furthermore, the existing contrast therapy apparatuses do not have a compressive therapy function.

### SUMMARY

A need exists for an integrated thermo- and cryo-therapy apparatus that can provide contrast therapy through a single device, which is also able to provide compressive therapy at the same time and is also portable so that a user carrying the apparatus can freely move during a treatment.

In one aspect, the present disclosure provides an apparatus for providing contrast and compression therapy, including: a hot water tank, a cold water tank, a water pump, a heat exchanger, and a therapy pad, where the hot water tank and the cold water tank are configured to store water at respective target temperatures for alternating delivery to the therapy pad for alternating thermotherapy and cryotherapy; a programmable control unit configured to automatically switch the alternating delivery of the stored water at the respective target temperatures to the therapy pad; and an air pump configured to deliver pressurized air to the therapy pad to provide a compression therapy simultaneously with the thermotherapy or the cryotherapy.

In some embodiments, the apparatus further includes a water reservoir tank connected to both the hot water tank and the cold water tank via a set of two-way valves, where the water reservoir tank facilitates water reuse between therapy sessions.

In some embodiments, the water reservoir tank is also fluidly connected to the heat exchanger and the water pump via one or more three-way valves, such that water from the reservoir tank is pumped through the heat exchanger to condition hot and cold water to the respective target temperatures before being delivered to the therapy pad.

In some embodiments, the apparatus operates without a water reservoir tank, and hot and cold water are supplied directly from external sources to the hot water tank and the cold water tank, respectively.

In some embodiments, the heat exchanger is disposed in a water flow path between the hot water tank or the cold water tank and the therapy pad and is configured to dynamically heat or cool water to the respective target temperatures during the contrast and compression therapy.

In some embodiments, the heat exchanger includes a temperature sensor configured to monitor a temperature of water flowing to the therapy pad and adjust the heating or cooling to maintain water at the respective target temperatures.

In some embodiments, the therapy pad includes at least two independent layers, including a fluid circulation layer for thermal therapy and an inflatable bladder for compression therapy.

In some embodiments, the water pump and the air pump are physically integrated into a single unit configured to operate in alternating or parallel modes.

In some embodiments, the hot water tank, the cold water tank, and optional water reservoir tank each include one or more hall effect sensors configured to monitor fluid levels within each tank.

In some embodiments, the apparatus further includes a water manifold configured to direct flow between multiple paths via a set of 2-way and 3-way valves, thereby forming closed-loop circulation paths for heating and cooling cycles.

In some embodiments, the water manifold is connected to the therapy pad, the water pump, and the air pump, and is configured to switch a direction of flow between thermotherapy and cryotherapy cycles without requiring manual intervention.

In some embodiments, the control unit is further configured to regulate a compression cycle timing.

In some embodiments, the apparatus further includes a detachable pad connector configured to interface with different therapy pads adapted for various anatomical regions.

In some embodiments, the apparatus is housed within a compact, portable casing and further includes a rechargeable power supply for self-contained operation.

In some embodiments, the rechargeable power supply is configured to provide power to the water pump, the air pump, the control unit, sensors, valves, and the heat exchanger without requiring an external power source during operation.

In some embodiments, dynamic temperature regulation is enabled through real-time feedback from the heat exchanger and the control unit.

In another aspect, the disclosure provides a system for delivering alternating thermotherapy and cryotherapy, including: a hot water tank, a cold water tank, a therapy pad, a heat exchanger, a plurality of valves, and a control unit, where the control unit is configured to: activate a water pump to direct water from the hot water tank through the heat exchanger and into the therapy pad for a first treatment cycle; subsequently activate one or more valves to route cold water from the cold water tank through the heat exchanger and into the therapy pad for a second treatment cycle; and repeat alternating cycles based on a user-defined or preset timing schedule for alternating thermotherapy and cryotherapy.

In some embodiments, the control unit further directs an air pump to supply pressurized air to a compressive layer of the therapy pad concurrently with delivery of hot or cold water, thereby applying compression in synchrony with the alternating thermotherapy and cryotherapy.

In some embodiments, water temperature is monitored by one or more sensors positioned within or adjacent to the heat exchanger, and the control unit adjusts heating or cooling output to maintain respective target temperatures throughout the first treatment cycle or the second treatment cycle.

In some embodiments, the apparatus operates in a reservoir-free configuration, and the control unit is further configured to flush fluid lines with air between treatment cycles to prevent mixing of residual thermal media, using solenoid-actuated valves to direct airflow through same circulation paths used for water delivery.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein can be employed in various and numerous embodiments.

### BRIEF DESCRIPTION OF THE DRAWINGS

The foregoing aspects and other aspects of the disclosure are described in detail below in connection with the accompanying drawings in which:

FIGS. 1A-1B illustrate various external views of an example contrast and compression therapy apparatus, according to some embodiments;

FIG. 2 illustrates an interval view of a partial of an example contrast and compression therapy apparatus, according to some embodiments;

FIG. 3 illustrates an example arrangement of an air pump and a valve and manifold assembly in a contrast and compression therapy apparatus, according to some embodiments;

FIG. 4A-4B illustrate different reviews of an example pad connector, according to some embodiments;

FIG. 5 illustrates an example arrangement of a water pump and a water manifold included in a contrast and compression therapy apparatus, according to some embodiments;

FIG. 6A illustrates a top view of an example water manifold, according to some embodiments;

FIG. 6B illustrates example water and air flow paths for the water manifold shown in FIG. 6A, according to some embodiments;

FIG. 6C illustrates example water and air flow paths for another water manifold, according to some embodiments;

FIG. 7 illustrates an internal view of a partial of an example valve and manifold assembly, according to some embodiments;

FIG. 8 illustrates an exploded view of an example valve and manifold assembly, according to some embodiments;

FIGS. 9A-9B illustrate various external views of an example hot/cold water tank, according to some embodiments;

FIG. 10 illustrates a transparent view of an example hot/cold water tank, according to some embodiments;

FIGS. 11A-11B illustrate various internal views of an example hot/cold water tank, according to some embodiments;

FIGS. 11C-11D illustrate various internal views of another example hot/cold water tank, according to some embodiments.

FIG. 12 illustrates a front view of an example water reservoir tank, according to some embodiments;

FIG. 13 illustrates a bottom view of an example water reservoir tank, according to some embodiments;

FIGS. 14A-14B illustrate different views of an example heat exchanger for a contrast and compression therapy apparatus, according to various embodiments;

FIG. 15A illustrates a flow diagram for water and air flow for a contrast and compression therapy apparatus, according to some embodiments;

FIG. 15B illustrates a flow diagram for water and air flow for another contrast and compression therapy apparatus, according to some embodiments;

FIG. 16 illustrates an example electronic system for a contrast and compression therapy apparatus, according to some embodiments; and

FIG. 17 illustrates a block diagram of an example computer system, according to various embodiments.

### DETAILED DESCRIPTION

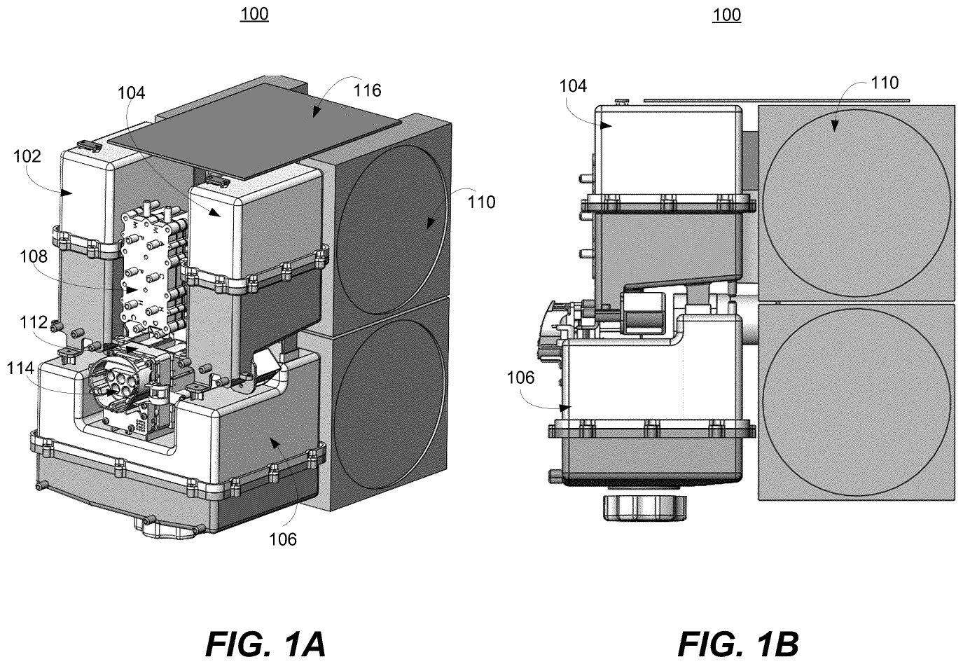

A contrast and compression therapy apparatus 100 is illustrated in FIGS. 1-17. As described below, the contrast and compression therapy apparatus 100 can be applied to different locations of the body. For example, the contrast and compression therapy apparatus 100 can apply alternating thermo- and cryo-therapy (also referred to as “contrast therapy”) to a selected location of the body, can apply compression to a selected location of the body, and can apply a combination of contrast therapy and compression therapy to a selected location of the body.

In some embodiments, when applying alternating thermo- and cryo-therapy, the contrast and compression therapy apparatus 100 disclosed herein can automatically switch between a thermotherapy section and a cryotherapy section without requiring human manipulation. For example, the disclosed contrast and compression therapy apparatus can automatically apply heat to a portion of the body under the treatment at one time point and automatically apply cold to the portion of the body under the treatment at another time point. The heat and cold can be automatically provided by the same contrast and compression therapy apparatus without requiring a user to manually switch between the heat source and the cold source. That is, in the disclosed contrast and compression therapy apparatus, both heat and cold can be generated and timely provided to the portion of the body under the treatment in a controlled manner. A user does not need to prepare different heat and cold sources. In addition, the heat and cold resources provided by the disclosed contrast and compression therapy apparatus do not elapse like many other existing contrast therapy apparatuses, such as apparatuses using ice or other types of cold sources.

In some embodiments, when applying heat and cold to a selected location of the body, the compression can be selectively applied by the disclosed contrast and compression therapy apparatus, to press the heat or cold into the selected location of the body, thereby increasing the efficiency of heat or cold delivery into the body. According to some embodiments, by providing compression while applying heat or cold to a selected location of the body, heat or cold can be delivered to a deeper part of the human body that cannot be reached without compression. For example, through combined heat and compression, heat can be delivered to a bone layer, which allows to convert tissue at the bone layer from gel to liquid viscosity. This can be accomplished by a combination of heat and pressure within certain bandwidth zones (e.g., high enough to achieve a therapeutic effect, but not so hot or intense as to hurt the person under the treatment). The other existing contrast therapy apparatuses cannot achieve such effects due to their limited functions of heat delivery into the human body.

According to some embodiments, the contrast and compression therapy apparatus 100 can be adapted to be used with wraps, which can be flexibly tied to different portions of the human body that have different shapes and sizes, even for certain portions of the human body that are generally not feasible for the existing heat wraps or hot pads. In addition, in some embodiments, the contrast and compression therapy apparatus 100 can be portable but does not require a user to stay close to a heat source and/or a cold source. A user under the treatment does not need to carry extra heat or cold source if a treatment lasts longer than expected. Therefore, a user under the treatment can easily carry the contrast and compression therapy apparatus 100 without considering additional elements or accessories for an ongoing treatment. The disclosed contrast and compression therapy apparatus is thus very convenient to use.

Referring to FIGS. 1A-1B, the contrast and compression therapy apparatus 100 includes a cold water tank 102 for holding cold water, a hot water tank 104 for holding hot water, and optionally a water reservoir tank 106 for holding water that can go into the cold water tank 102 or hot water tank 104, or water drained from the cold water tank 102 and/or hot water tank 104. In some embodiments, to set up proper circulations of hot/cold water, a water manifold 108 is further included in the contrast and compression therapy apparatus 100.

In some embodiments, a set of heat exchangers 110 are also included in the apparatus 100, which can be controlled to heat or cool water passing through it to a certain degree to obtain cold water or hot water for contrast therapy. In other words, through the same set of heat exchangers 110, both hot and cold water can be produced in the disclosed contrast and compression therapy apparatus 100.

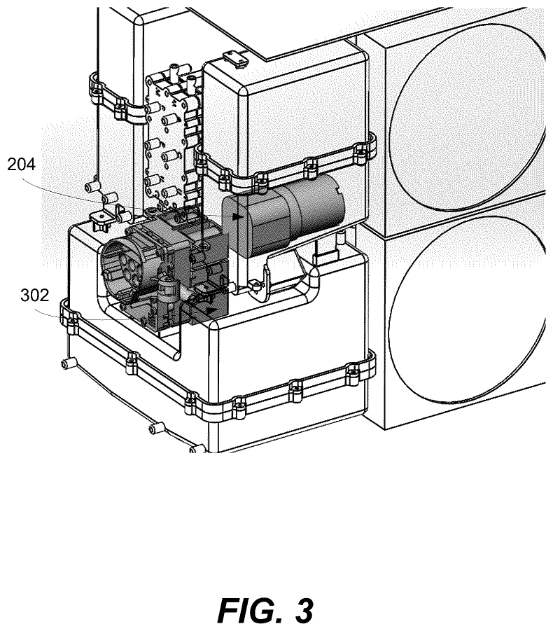

In some embodiments, the contrast and compression therapy apparatus 100 additionally includes an air manifold 112 (which is part of a valve and manifold assembly, as will be described in FIG. 3), which is an air distribution manifold used to divert a single gas, air, or liquid feed line to multiple locations or devices. Although not specifically illustrated, the contrast and compression therapy apparatus 100 further includes an air pump connected to the air manifold 112. For one purpose, the air pump can pump air into one or more circulation loops built upon the 2-way and 3-way valves, the connection tubings, and the water manifold to empty the hot/cold water included in the tubings so that hot water and cold water can be alternatively provided to a therapy pad connected to the contrast and compression therapy apparatus 100.

In some embodiments, the contrast and compression therapy apparatus 100 also includes a pad connector 114 for connecting a therapy pad to the therapy apparatus. As will be described in detail, more than one kind of therapy pad can be connected to the same contrast and compression therapy apparatus 100. Accordingly, by connecting to different therapy pads, the contrast and compression therapy apparatus 100 can be applied to many different application scenarios.

It should be noted that, in some embodiments, a therapy pad can itself include a heat (or cold) generation unit such as a heater, and thus only the cold (or hot) water tank and the corresponding cryotherapy are functional in the contrast and compression therapy apparatus disclosed herein. The hot (or cold) water tank can remain isolated from the circulation system under such circumstances.

In some embodiments, the contrast and compression therapy apparatus 100 further includes a printed circuit board (PCB) control unit 116. The specific operations of the contrast and compression therapy apparatus 100 can be controlled by the PCB control unit 116 for automated operation without requiring much human manipulation during treatments. For example, the PCB control unit 116 can automatically control the switches of different 2-way and 3-way valves included in the contrast and compression therapy apparatus 100. In addition, the PCB control unit 116 also controls the heat exchangers 110 to respond to a proper temperature set for heating or cooling the water when necessary. Further, the PCB control unit 116 also controls the compression applied to a therapy pad, e.g., through controlling the air pump, as will be described in detail later.

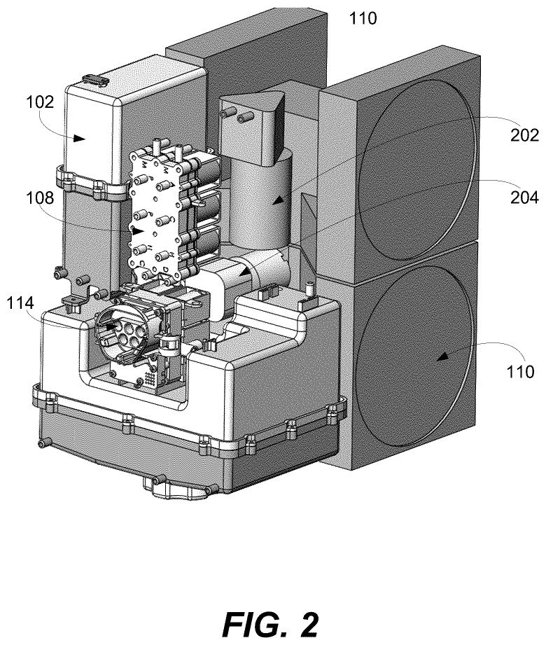

Referring now to FIG. 2, an internal view of a partial of the contrast and compression therapy apparatus 100 is further illustrated. As illustrated in the figure, the contrast and compression therapy apparatus 100 includes a water pump 202 and an air pump 204. The water pump 202 is disposed between the cold water tank 102 and the hot water tank (not shown), and between the water manifold 108 and the heat exchanger 110. The air pump 204 is disposed below the water pump 202, as can also be seen in the figure. Although not shown in FIG. 2, the water pump 202 is connected to the cold water tank, cold water tank, and the optional water reservoir tank through a set of 2-way and/or 3-way valves.

The water pump 202 is the driving unit that drives the water circulation inside the contrast and compression therapy apparatus 100. For example, the water pump 202 drives the water to flow from the water tank reservoir to the cold water tank or hot water tank (water can be heated or cooled by the heat exchangers during the process). The water pump 202 also drives cold water to flow from the cold water tank to a therapy pad during a cryotherapy section, and drives hot water to flow from the hot water tank to the therapy pad during a thermotherapy section (the water can also be heated or cooled during each section if the hot water or cold water fells a little below or above the target temperature).

The water pump 202 can be an electrical pump powered by electricity (e.g., backed by the battery). In addition, different types of pumps can be used in the contrast and compression therapy apparatus 100. Example water pumps include, but are not limited to, positive displacement pump, rotary displacement pump, dynamic pump, centrifugal pump, axial & radial centrifugal pump, reciprocating pump, peristaltic pump, etc.

In some embodiments, right after completion of the whole treatment, the hot or cold water can be pumped back from the cold water tank 102 and the hot water tank 104 to the water tank reservoir 106 for readiness to be used in the next treatment. In some embodiments, water in the cold water tank 102 or hot water tank 104 can remain in these tanks in case a new treatment will start soon, which then saves the time and energy to initiate the hot water tank or cold water tank again. For example, the hot water in the hot water tank can be used for initiation of the hot water tank (e.g., through proper switches of 2-way and 3-way valves) to get the desired amount and/or temperature of hot water in the tank. In situations where the expected volume of hot water for the next treatment is larger than the current volume in the tank, a certain volume of water can be drawn from the water reservoir tank and heated through the heat exchangers, to get the desired amount of water during the “re-heat” process. On the other hand, if the expected volume of hot water is less than the current volume in the tank, a certain volume of water can be pumped back or drained to the water tank reservoir 106 before the “re-heat” process. It should be noted that the “re-heat” process does not necessarily mean an increase of the hot temperature in the tank. Depending on the purposes of applications, if the expected temperature for the next treatment is lower than the temperature of hot water already in the hot water tank, the “re-heat” process can lower the temperature through directing the hot water to pass through the heat exchangers again, since the heat exchangers can both increase or decrease water temperature through the heat exchange process.

The air pump 204 is the driving unit that pumps air into or out of the contrast and compression therapy apparatus 100 when emptying the tubings or water residuals in the valves when switching between a thermotherapy section and a cryotherapy section. For example, after a thermotherapy section, the water in the tubings and the therapy pad can be blown back to the hot water tank through the air pump 204 (alternatively, it can also be pumped through the water pump 202). This then allows cold water to be pumped into the tubings and further into the therapy pad for the following cryotherapy section. After the cryotherapy section, the water in the tubings and the therapy pad can be similarly blown back to the cold water tank through the air pump 204 (alternatively, it can also be through the water pump 202), to prepare the contrast and compression therapy apparatus 100 for the following thermotherapy section in the alternating thermo- and cryo-therapy.

In some embodiments, the air pump 204 is also connected to the therapy pad through the air manifold 112 to apply proper pressure during the compression therapy under certain application scenarios. In some embodiments, a therapy pad includes a layer for compression and another layer for thermotherapy and cryotherapy. The compression layer is disposed on a side away from the skin, while the layer for the thermotherapy and cryotherapy is disposed on a side adjacent to the skin. The compression layer can press the heat or cold into the body during a treatment to increase the delivery of the heat or cold for improved efficiency. In some embodiments, the compressed heat or cold can reach a deeper part of the tissue or organs, which can produce a treatment effect not available for a thermo- and cryo-therapy pad without compression.

In some embodiments, the air pump 204 can continuously adjust the air pressure within the inflatable portion of the compression layer of the therapy pad, so as to achieve preset internal air pressure within the inflatable portion in order to achieve the optimal pressure. This process is accomplished by continuously adding or removing air from the inflatable portion to adjust to the varying load placed on the layer of thermo- and cryo-therapy. In some embodiments, the air pump 204 is connected to the therapy pad through a valve and manifold assembly, as further described in detail in FIG. 3.

Referring to FIG. 3, a transparent view of an arrangement of the air pump 204 and the valve and manifold assembly 302 is further illustrated, according to some embodiments. The valve and manifold assembly 302 is an assembly of the air manifold 112 for air distribution and 2-way and 3-way valves for water or air flow. In some embodiments, the water valves and air manifolds in the valve and manifold assembly 302 each can be independently controlled by a control unit such as the PCB control unit 116. For example, the 2-way and 3-way valves included in the valve and manifold assembly 302 can be controlled to automatically rotate to a specific direction based on the signal generated by the PCB control unit 116. Similarly, the air manifolds in the valve and manifold assembly 302 can be controlled to automatically charge or discharge air based on the signal generated by the PCB control unit 116. In this way, the PCB control unit can control the operation of the whole valve and manifold assembly 302 by controlling the valves and air manifolds included in the valve and manifold assembly 302. For example, through proper control of water valves in the valve and manifold assembly 302, a hot water buildup loop, a cold water buildup loop, a thermotherapy circulation loop, and a cryotherapy circulation loop can be respectively formed (or disabled). Similarly, through control of air manifolds in the valve and manifold assembly 302, air can be distributed to the therapy pad at one moment, and distributed to the tubings for dispelling water included in a circulation loop or path at another moment.

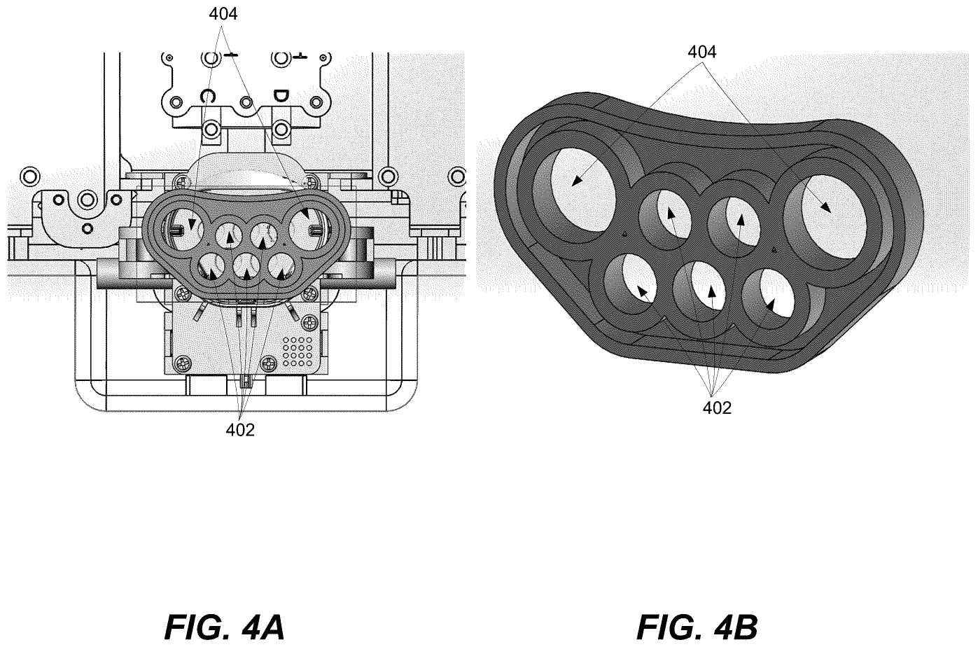

In one example, there are two 2-way valves and five 3-way valves included in the valve and manifold assembly 302, as will be described in FIGS. 4A-4B. In another example, the exact number of 2-way valves and 3-way valves included in the assembly can vary, depending on the number of zones included in a therapy pad. In real applications, the exact number of air manifolds included in the valve and manifold assembly 302 can also vary and depend on the specific configurations (e.g., how many pad zones and the specific pressure requirement of these zones).

It should be noted that while the valve and manifold assembly 302 is illustrated as a single piece or integrated unit, in some embodiments, the valves can form a valve assembly and the manifolds can form another different manifold assembly, which can be disposed at an adjacent location of the valve assembly or can be disposed at a location away from the valve assembly, which is not limited in the present disclosure.

In some embodiments, the valve and manifold assembly 302 is disposed between the air pump 204 and the pad connector 114, as can be seen from FIG. 3. Through the pad connector 114, the water and air can be pumped into the therapy pad in a predefined manner.

Referring to FIG. 4, a specific configuration of the pad connector 114 is illustrated, according to some embodiments. As illustrated in the figure, the pad connector 114 includes a number of through-holes, through which the water tubings and air tubings can be guided into different sections of a therapy pad. In the example embodiment shown in FIG. 4, there are five air connections 402 and two water connections 404. The two water connections 404 each correspond to a tubing for flowing water into a therapy pad, and a tubing for flowing water out of the therapy pad. As described elsewhere herein, thermotherapy and cryotherapy are alternated, and thus, at any moment, there is only hot water or cold water pumped into the therapy pad.

It should be noted that the pad connector 114 shown in FIG. 4A is an enlarged view when compared to other parts of the contrast and compression therapy apparatus 100 shown in the figure. In addition, the alignment and the shape of the air connections and water connections are not limited to the configuration shown in FIGS. 4A-4B. For example, the two water connections are not necessarily disposed on two corners of the top part of the pad connector 114, but rather can be disposed at the bottom part of the pad connector 114. In addition, while the water connections 404 and air connections 402 are illustrated to have a circular shape, these connections can have other different shapes, such as elliptical, square, or other proper shapes.

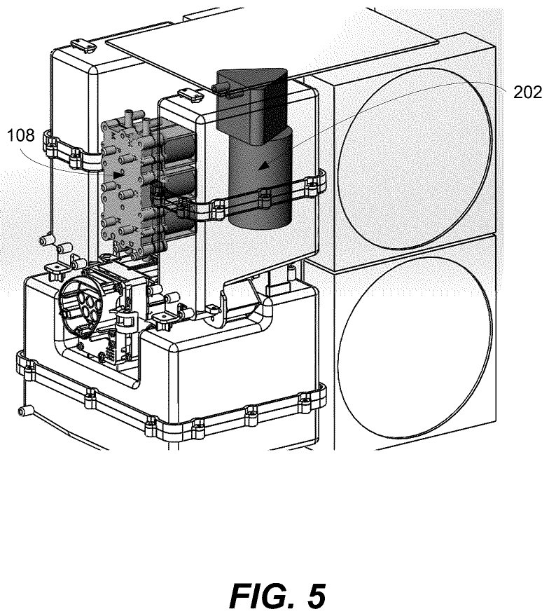

Referring to FIG. 5, a transparent view of an arrangement of the water pump 202 and the water manifold 108 is further illustrated, according to some embodiments. As illustrated in the figure, the water pump 202 is disposed behind the water manifold 108 (the side where the therapy pad is connected is considered to be the front side and the opposite side is considered to be the back side) and between the water manifold 108 and the heat exchanger 110. As can also be seen in FIGS. 2 and 5, the water pump 202 is also disposed between the PCB control unit 116 and air pump 204.

In some embodiments, the water pump 202 is connected to the heat exchanger(s) 110 through the tubings, so that when the water pump 202 is driven to run, the water pump 202 also drives the water in the tubings to pass through the heat exchanger(s) 110 to heat or cool down the water to a certain temperature when the hot water/cold water is built up in the respective tank, or to maintain the water in the tubings at a desired (high or low) temperature during a thermotherapy section or cryotherapy section.

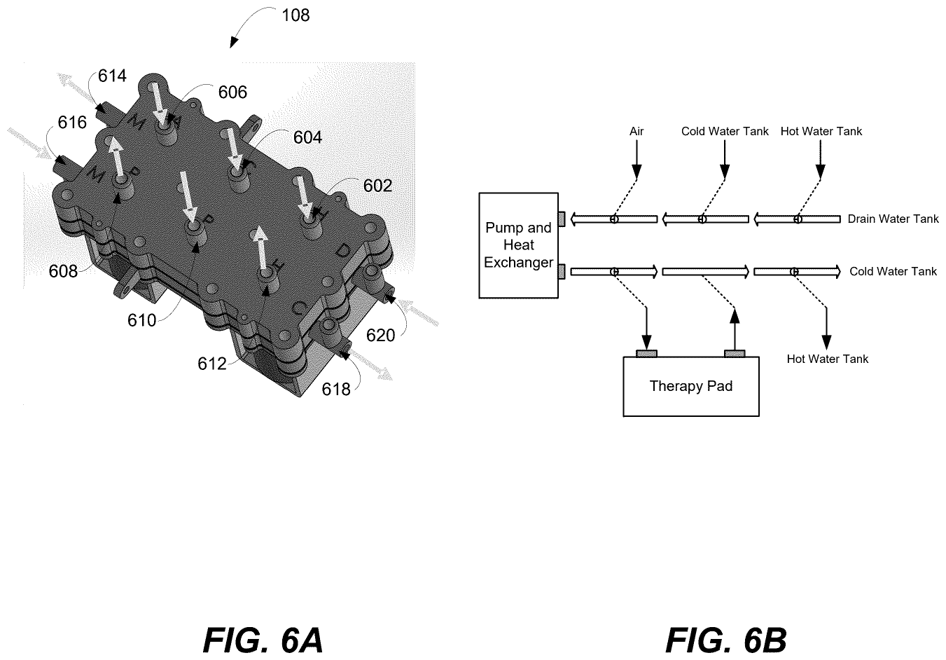

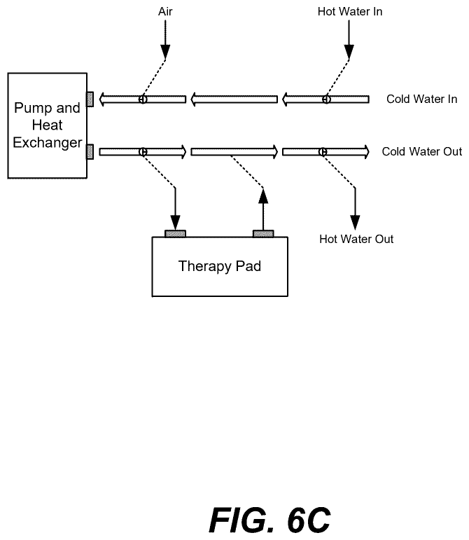

Referring to FIGS. 6A-6B, an example structure of a water manifold and water and air flows associated with the water manifold are illustrated, according to some embodiments. Specifically, for the water manifold 108, there are six ports popped out of the top surface illustrated in FIG. 6A. Specifically, the six ports include a first port 602 connected to the hot water tank to allow hot water to be pumped into the water manifold 108 through a controlled 3-way valve (not shown), a second port 604 connected to the cold water tank to allow cold water to be pumped into the water manifold 108 through another controlled 3-way valve (not shown), a third port 606 connected to the air pump (not shown) through an air tubing to allow air to be pumped into the water manifold 108 (e.g., to expel water in the water manifold after a thermotherapy section or cryotherapy section is completed), a fourth port 608 connected to the therapy pad to allow hot/cold water as well as air to be bumped into the therapy pad during a treatment, a fifth port 610 also connected to the therapy pad to allow the hot/cold water as well as air to be pumped out of the therapy pad during a treatment, and a sixth port 612 connected to the hot water tank to allow hot water to be pumped back to the hot water tank during a thermotherapy section or when the hot water is built up in the hot water tank in the initiation stage of a treatment.

As also illustrated in FIGS. 6A-6B, on one side surface of the water manifold 108 (e.g., the left side shown in FIG. 6A), there are two additional ports for connection to the pump and heat exchanger. For example, there is a seventh port 614 for connection to the water/air pump and heat exchanger to allow water to pass through the heat exchanger during a thermotherapy or cryotherapy section and to allow air to flow along the water manifold 108. As can be seen in FIG. 6A, there is also an eighth port 616 connected to the water/air pump and heat exchanger to allow water or air to return to the water manifold 108.

As further illustrated in FIGS. 6A-6B, on an opposite side surface of the water manifold (e.g., the right side shown in FIG. 6A), there are also two ports for connection to the cold water tank and the water reservoir tank. Specifically, there is a ninth port 618 connected to the cold water tank for water passing through the water manifold 108 to be pumped back to the cold water tank. In addition, there is also a tenth port 620 connected to the water reservoir tank for drawing water from the reservoir when the hot/cold water is built up during the initiation stage.

In the following, how water or air passes through the water manifold 108 is further illustrated by taking a few example applications. Specifically, in one example application for building up water in the hot water tank during the initialization stage, water is drawn from the water reservoir tank through the port 620, and the drawn water flows inside the water manifold 108 along one side (e.g., upper side in FIG. 6A) of the water manifold before being pumped out of the water manifold through the port 614 and into the heat exchanger for heating the water to a target degree. The heated water then returns to the water manifold 108 through the port 616, where the water flows inside the water manifold 108 along the other side (e.g., lower side in FIG. 6A) of the water manifold before being directed out of the port 612, which is connected to the hot water tank. In this way, water can be drawn from the water reservoir tank and heated by the heat exchanger and then the heated water is pumped into the hot water tank until a target amount of water is obtained in the hot water tank. Once the heated water is sufficient, the water is not drawn from the water reservoir tank anymore. Air is then drawn into the water manifold 108 through the port 606, to expel the remaining water inside the water manifold 108 into the hot water reservoir. It should be noted that there is a small amount of unheated water (e.g., water on the upper side of the water manifold connected to the water reservoir tank) expelled into the hot water tank, which can affect the temperature of water in the hot water tank. However, since the hot water in the hot water tank will run through the heat exchanger again during a thermotherapy section, the water in the hot water tank can be dynamically controlled during a thermotherapy section (e.g., based on a thermometer or another type of sensor coupled to the heat).

In another example application, cold water can be built up in the cold water tank during the initialization stage. Specifically, water is drawn from the water reservoir tank through the port 620. The drawn water flows inside the water manifold 108 along one side (e.g., upper side in FIG. 6A) of the water manifold before being pumped out of the water manifold through the port 614 and into the heat exchanger for cooling the water down to a target degree. The cooled water then returns to the water manifold 108 through the port 616, where the water flows inside the water manifold 108 along the other side (e.g., lower side in FIG. 6A) of the water manifold before being directed out of the port 618, which is connected to the cold water tank. In this way, water can be drawn from the water reservoir tank and cooled by the heat exchanger and then the cooled water is pumped into the cold water tank until a target amount of water is obtained in the cold water tank. Once the cooled water is sufficient, the water is not drawn from the water reservoir tank anymore. Air is then drawn into the water manifold 108 through the port 606, to expel the remaining water inside the water manifold 108 into the cold water reservoir. It should be noted that there is a small amount of uncooled water (e.g., water on the side of the water manifold connected to the water reservoir tank) expelled into the cold water tank, which can affect the temperature of water in the cold water tank. However, since the cold water in the cold water tank will run through the heat exchanger again during a cryotherapy section, the water in the cold water tank can be dynamically controlled during a cryotherapy section (e.g., based on a thermometer or another type of sensor coupled to the heat exchanger).

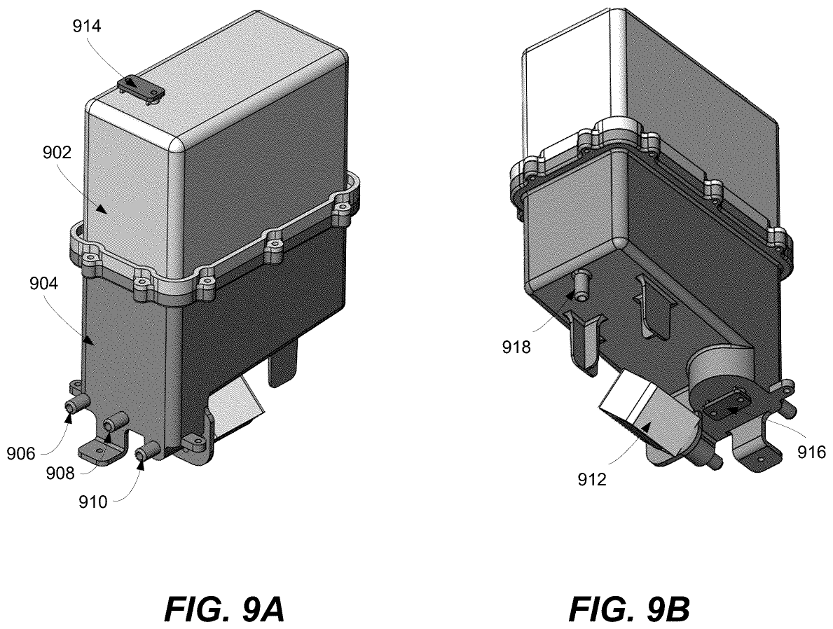

In another example application for a thermotherapy section, hot water is drawn (e.g., through the water pump) into the water manifold 108 through the port 602. The hot water flows through one side (e.g., upper side in FIG. 6A) of the water manifold before being drawn into the heat exchanger. In the heat exchanger, the water is heated again if necessary before being pumped back to the water manifold 108 through the port 616. The hot water then runs along the other side (e.g., lower side in FIG. 6A) of the water manifold before being directed to the therapy pad through the port 608. After passing through the therapy pad, the hot water flows back to the water manifold 108 through the port 610, and further into the hot water tank through the port 612. It should be noted that while the port 602 and the port 612 are disposed on two opposite sides of the water manifold 108, both ports are connected to the hot water tank through different tubings connected to two different ports of the hot water tank, as will be described later in FIGS. 9A-9B. In some embodiments, after completion of a thermotherapy section, the water remaining in the water manifold is blown out of the water manifold 108 and into the hot water tank through the port 612, as described earlier.

In yet another example application for a cryotherapy section, cold water is drawn (e.g., through the water pump) into the water manifold 108 through the port 604. The cold water flows through one side (e.g., upper side in FIG. 6A) of the water manifold before being drawn into the heat exchanger. In the heat exchanger, the water is cooled again if necessary before being pumped back to the water manifold 108 through the port 616. The cold water then runs along the other side (e.g., lower side in FIG. 6A) of the water manifold 108 before being directed to the therapy pad through the port 608. After passing through the therapy pad, the cold water flows back to the water manifold 108 through the port 610, and further into the cold water tank through the port 618. It should be noted that while the port 604 and the port 618 are disposed on two different surfaces of the water manifold 108, both ports are connected to the cold water tank through two different tubings connected to two different ports of the cold water tank, as will be described later in FIGS. 9A-9B. In some embodiments, after completion of a cryotherapy section, the water remaining in the water manifold is blown out of the water manifold 108 and into the cold water tank through the port 618, as described earlier.

It should be noted that, while the 2-way and 3-way valves are involved in controlling the water and/or air flow in the disclosed contrast and compression therapy apparatus 100, the additional mechanisms for controlling the air or water flow inside the water manifold 108 are possible and contemplated in the present disclosure.

In some embodiments, a water reservoir tank may not be included in the disclosed contrast and compression therapy apparatus 100, as described earlier. Under such circumstances, the water manifold 108 may have a different configuration than that illustrated in FIGS. 6A-6B. FIG. 6C illustrates another example water manifold along with its air and water flow paths, according to some embodiments. Compared to the water manifold illustrated in FIGS. 6A-6B, the water manifold in FIG. 6C does not connect to a water reservoir tank described above. Accordingly, there are only ports for getting respective cold water and hot water into and out of the water manifold.

Specifically, the diagram in FIG. 6C represents a streamlined fluid water manifold designed to operate without a water reservoir tank, utilizing direct hot and cold water supply lines in conjunction with solenoid-controlled valves, a pump and heat exchanger, and a therapy pad. The water manifold features distinct fluid pathways for cold water, hot water, and compressed air, all of which converge at a central manifold and are directed toward or away from the therapy pad depending on the treatment phase.

In operation, cold water enters the water manifold from a designated inlet (connected to the cold water reservoir) on the right side of the upper flow path. A solenoid valve regulates the admission of cold water, which then flows laterally toward the pump and heat exchanger unit. This unit either directly circulates the water or adjusts its temperature as needed before directing it downward to the therapy pad. Once inside the therapy pad, the cold water provides localized cryotherapy to the patient. After the treatment cycle, the used cold water exits the therapy pad through a separate outlet and is directed horizontally across the lower flow path to the cold water out connection (also connected to the cold water reservoir), which together forms a cold water circulation loop during the treatment cycle for cryotherapy.

The hot water cycle functions in a similar manner. Hot water enters the system via its own solenoid-controlled inlet (connected to the hot water reservoir) and is routed through the same pump and heat exchanger unit. From there, the water is delivered to the therapy pad through a separate entry point, allowing thermotherapy to be administered. The spent hot water exits the pad through a dedicated outlet and follows a separate discharge line to the hot water out connection (also connected to the hot water reservoir), which together forms a hot water circulation loop during the treatment cycle for thermotherapy.

Compressed air enters the system through an independent solenoid valve positioned at the far left of the upper flow path. The air can be routed through the same tubing infrastructure as the water, enabling it to purge any residual fluid between hot and cold therapy cycles. This flushing process prevents mixing of thermal media and prepares the system for the next mode of operation. Additionally, the air may be used to inflate a compression layer within the therapy pad, thereby enhancing therapeutic effectiveness by pressing the heat or cold deeper into the tissue.

Together, these independent but coordinated flow paths enable the apparatus to perform alternating hot and cold treatments with high efficiency and no internal water storage. The absence of a reservoir reduces system complexity, minimizes maintenance, and enables a more compact and portable design. All fluid routing decisions are handled through a programmable control unit that activates the appropriate solenoid valves and coordinates the timing of pump operation and heat exchange, ensuring seamless transitions between therapy modes.

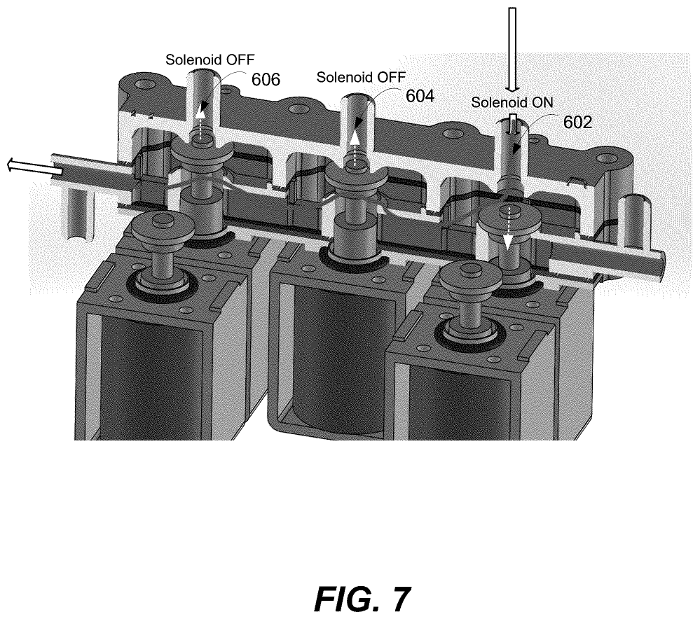

Referring to FIG. 7, an example mechanism for controlling water or air flow inside the water manifold is illustrated, according to some embodiments. As illustrated in the figure, for a port disposed on the top surface(s) of the water manifold, there can also be an associated solenoid valve, which can be controlled to turn on or off through a controlling mechanism. That is, wherever fluid flow has to be controlled automatically, a solenoid valve is used in the corresponding place. Solenoid valves are generally electric-actuated on-off valves, which can have a compact design. Some models are immune to mounting orientation. Solenoid valves are compatible with a wide range of gases and liquids, and the applications of these solenoid valves include flow control, water, process control, and chemical mixing, among others. In the water manifold 108 disclosed herein, different types of solenoid valves can be used, which are not limited in the present disclosure.

Referring back to FIG. 7, when a solenoid valve associated with the port 602 is actuated (e.g., the cap is pulled down), the port is opened to the water manifold so that the hot water can be drawn into the water manifold through the port 602. Since cold water and air are not expected to be drawn into the water manifold, the solenoid valves associated with the ports 604 and 606 are actuated off (e.g., caps are pushed up), which prevents the air and cold water from being drawn into the water manifold 108. It should be noted that FIG. 7 only illustrates one example application for controlling the solenoid valves associated with the ports 602, 604, and 606, the control of solenoid valves for other applications can be similarly obtained, which are not specifically described in the present disclosure.

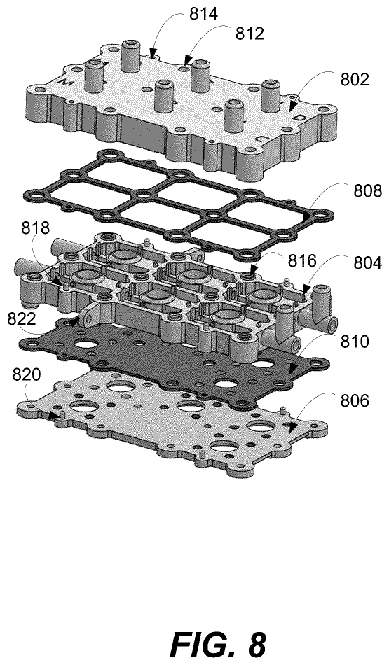

Referring now to FIG. 8, an exploded view of a specific structure of the water manifold is further illustrated, according to some embodiments. As illustrated, the water manifold includes three plastic pieces 802, 804, and 806, and two seal rubbers 808 and 810. The three plastic pieces include a first piece 802 as the top cover piece, a second piece 804 as the main frame piece, and a third piece 806 as the bottom cover piece. The six ports 602-612 disposed on the top surface of the water manifold can be formed together with the top cover piece 802 (e.g., through a same molding process), and the four ports 614-620 disposed on two side surfaces of the water manifold 108 can be formed together with the main frame piece 804 (e.g., through a same molding process).

As further illustrated in FIG. 8, besides the six ports 602-612, the top cover piece 802 additionally includes a number of bores 812 for inserting screws for holding the five pieces 802-810 together as a single manifold. In the illustrated embodiment, there are twelve bores 812 aligned in four rows, with each row including three bores. Besides the bores for inserting the screws, the top cover piece 802 additionally includes a set of mounting bores 814 for aligning different pieces (e.g., aligning the main frame piece 804 with the top cover piece 802). In the illustrated embodiment in FIG. 8, there are four mounting bores 814 for mounting the top cover piece 802 onto the main frame piece 804. For example, there is a mounting bore disposed at an edge close to each of the ports 602, 606, 608, and 612. In some embodiments, there are certain markers (such as H, C, A, M, M, P, P, H, C, and D) for easier identification of the specific ports. Here, “H” represents the hot water tank connection, “C” represents the cold water tank connection, “A” represents the air tubing connection, “M” represents the pump and heat exchanger connection, “P” represents the therapy pad connection, “D” represents the drain connection or the water reservoir tank connection.

It should be noted that while not shown, the bottom part of the top cover piece 802 is not flat but rather has a concaved shape. For example, as can be seen further in FIG. 7, there are three concaved portions disposed on one side (e.g., upper side) of the water manifold 108. While not shown in FIG. 7, there are another three concaved portions on the other side (e.g., lower side) of the water manifold 108, so that there are six concaved portions in total in the disclosed water manifold. The six concaved portions of the top cover piece 802, when assembled together with the main frame piece 804 and the bottom cover piece 806, will form six separated chambers.

With respect to the middle main frame piece 804, it includes a set of through holes 816 to allow the screws to pass through the middle main frame piece. In the illustrated embodiment in FIG. 8, there are twelve through holes 816, which is the same as the number of bores in the top cover piece 802. As further illustrated in FIG. 8, there are a set of mounting bars 818, which correspond to the mounting bores 814 disposed in the top cover piece 802. It should be noted that, while not shown, the bottom part of the main frame piece 804 additionally includes a set of mounting bores, which corresponds to a set of mounting bars 820 disposed on the top surface (facing the main frame piece) of the bottom cover piece 806.

As also can be seen in FIG. 8, besides the edge frames aligned along the four edges of the water manifold, there are additional inner vertical frames in the main frame piece 804 to separate the main frame piece into six chambers, each chamber corresponding to one of the six ports 602-612. Each chamber includes a central column for mounting a corresponding solenoid valve (not shown). Each central column is fixed by two vertical blocks aligned along the same direction. One of the two vertical blocks has a top surface flush with the top surface of the inner vertical frames, while the other one of the two vertical blocks has a top surface that is lower than the top surface of the inner vertical frames. The lower vertical block can allow water or air to freely flow within each chamber. As can also be seen in FIG. 8, the shorter one of the two vertical blocks has a width narrower than the other one of the two vertical blocks.

Although not shown, in some embodiments, between two adjacent chambers aligned along one direction (e.g., aligned along a direction from port 620 to port 614), there is a through hole, through which water and air can freely flow from one chamber to another. In addition, for each corner chamber (such as chambers corresponding to ports 602, 606, 608, and 612), there is also a hole between the chamber and the corresponding side port (e.g., a hole between the port 614 and the chamber corresponding to the port 606). This also allows water or air to flow into or out of the water manifold through the corresponding port.

As further shown in FIG. 8, for the seal rubber 808 between the top cover piece 802 and the middle main frame piece 804, the seal rubber 808 is also divided into six sections corresponding to six chambers. The seal rubber 808 prevents water or air from flowing from the top surface of each chamber to an adjacent chamber. The seal rubber 808 also includes 12 through holes for allowing screws to pass through and four through holes to allow the mounting bars to pass through. For the bottom rubber seal 810, it will seal the six chambers from the bottom. In some embodiments, the bottom rubber seal 810 also includes six big holes (while only five are shown in the figure) that allow the solenoid valves to pass through. Similar to the rubber seal 808, the rubber seal 810 also includes 12 through holes to allow the screws to pass through and four through holes to allow the mounting bars to pass through.

With respect to the bottom cover piece 806, it has a shape similar to the rubber seal 810 except that it includes four mounting bars 820 that the rubber seal 810 does not have. In addition, the 12 bores in the bottom cover piece 806 can also be threaded to receive screws inserted from the top cover piece. In some embodiments, the bores in the bottom cover piece 806 may not be threaded. Instead, hex-nuts are used to fix the screws to the bottom cover piece 806, which is not limited in the present disclosure.

In some embodiments, the water manifold 108 includes additional components not described above. For example, the main frame piece 804 can include two protrusions 822 aligned along two opposite sites, where each protrusion includes a bore for allowing the whole water manifold 108 to be fixed to certain other components of the contrast and compression therapy apparatus 100 through screws or through other fixing mechanisms.

Referring now to FIGS. 9A-9B, side and bottom views of an example structure of a hot or cold water tank are illustrated, according to some embodiments. It should be noted that in the disclosed contrast and compression therapy apparatus 100, the hot water tank and cold water tank can have the same structure since both are used to hold water at a specific temperature.

As illustrated in FIG. 9A, the hot/cold water tank includes two separate parts 902 and 904 that are held together through a set of screws around the tank. For the bottom part 904, it further includes a number of ports for connection to different components. For example, there is a first port 906 for connection to the water manifold 108, a second port 908 for connection to the water manifold 108, and a third port 910 for connection to the water reservoir tank or a hot/cold water source (or water input). For example, the first port 906 can allow water to be pumped into the hot/cold water tank through the port, the second port 908 can allow water to be pumped out of the hot/cold water tank through the port, and the third port 910 can allow water to be obtained from the water source, which may timely provide hot or cold water on-demand and/or at a predefined frequency.

As further illustrated in FIG. 9B, the hot/cold water tank also includes a drain solenoid valve 912 connected to the port 910. The drain solenoid valve 912 is mainly used to control water to be drained to the water reservoir tank when the water reservoir tank is included in the apparatus.

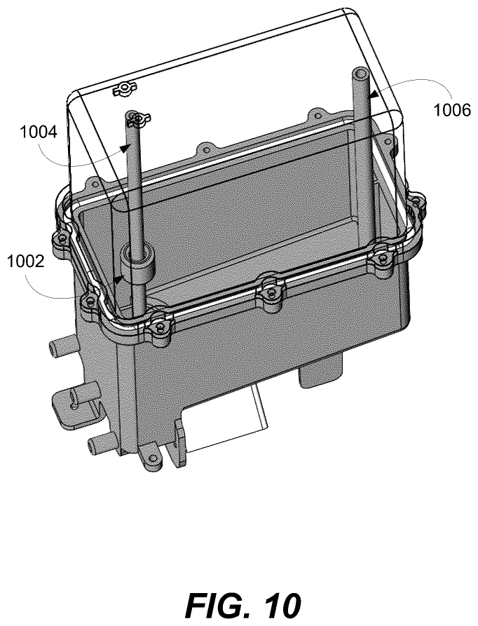

In some embodiments, the hot/cold water tank includes an additional drain port, through which the water overflow can be drained to the water reservoir tank if it is included in the apparatus, as further illustrated in FIG. 10.

In some embodiments, the material used to make the hot/cold tank can be plastic. In some embodiments, certain insulation material is injected into the walls of the container to increase thermal insulation. In some embodiments, the hot/cold water tank further includes certain accessory components for fixing or attachment of the hot/cold water tank to other components included in the contrast and compression therapy apparatus 100, or allow other components to attach to the hot/cold water tank. Example accessory components include but are not limited to certain protrusions disposed at different locations and/or having different shapes. In some embodiments, these protrusions include one or more holes allowing screws to be used for purposes of securing different components.

In some embodiments, the hot/cold water tank further includes a top hall sensor 914 disposed on the top of the hot/cold water tank and a bottom hall sensor 916 disposed at the bottom of the hot/cold water tank. The top hall sensor 914 and the bottom hall sensor 916 are configured to measure the water level in the tank so that the volume of hot/cold water can be timely determined. A hall sensor (also known as a hall effect sensor or hall probe) is a sensor incorporating one or more hall elements, each of which produces a voltage proportional to one axial component of a magnetic field vector using the hall effect.

In the embodiment illustrated in FIG. 10, the axial component of the magnetic field vector is a magnetic float 1002 that moves when the water level in the hot/cold water tank fluctuates. In the illustrated embodiment, the magnetic float 1002 is a magnetic ring that freely moves along a vertical bar 1004. Since the magnetic ring moves with the moving water surface in the hot/cold water tank, this magnet displacement distance is the same as the linear position displacement of the moving water surface.

In some embodiments, for the hall sensor-based distance measurement, a linear hall position sensor is used to detect the distance at multiple points along the travel path of the magnetic vector. Accordingly, there are two hall sensors 914 and 916 disposed along the travel path of the magnetic ring (e.g., the hall sensors 914 and 916 are disposed at positions aligned with the vertical bar 1004 to form a straight line).

In some embodiments, the hot/cold water tank further includes an overflow pipe 1006 that allows the water to be drained to the water reservoir tank in case there is too much water being pumped into the hot/cold water tank. In some embodiments, at the bottom of the hot/cold water tank, there is also a port 918 connected to the overflow pipe for draining the overflow water to the water reservoir tank. In some embodiments, the vertical bar 1004 can also be multiplexed as the overflow pipe 1006 to simplify the manufacturing process of the hot/cold water tank.

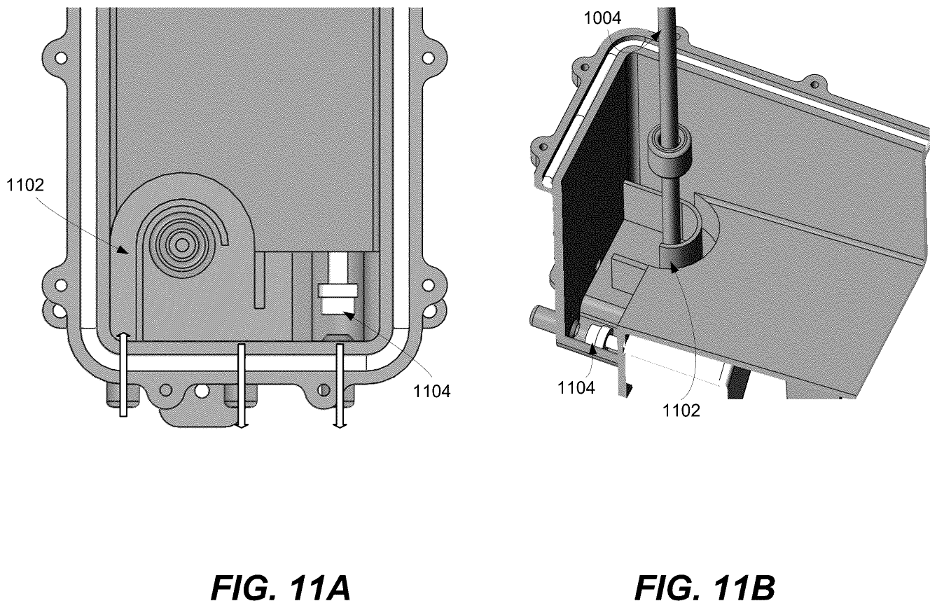

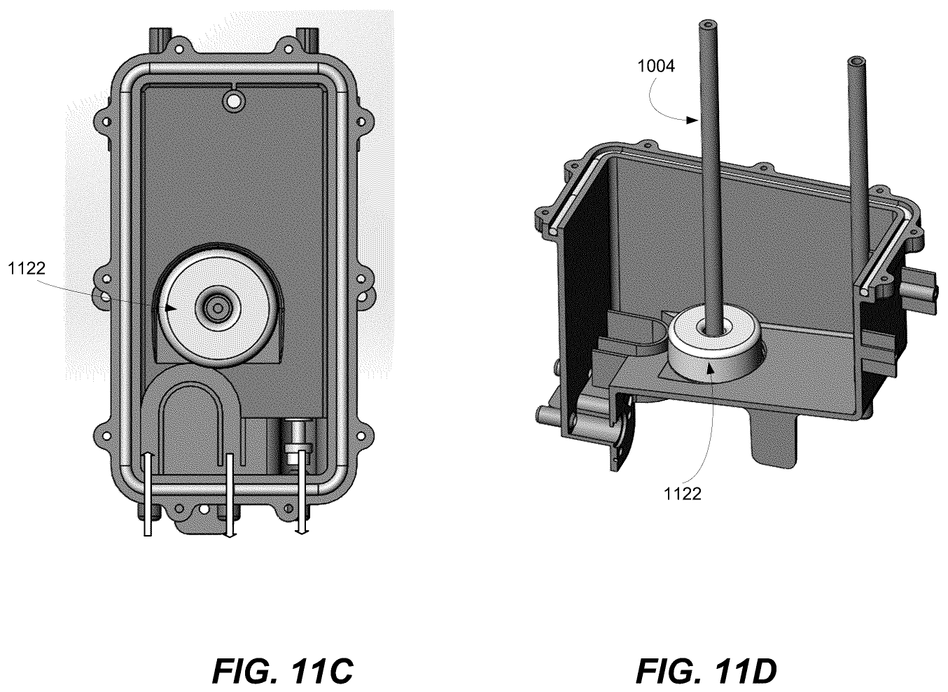

Referring now to FIGS. 11A-11B, the internal structure of the bottom part of the hot/cold water tank is further illustrated. As shown in the figures, at the bottom part of the hot/cold water tank, there is a water return guide 1102, which is specifically configured to be a vertically aligned roller-shape tunnel with a straight section and a curved section. The straight section starts from a corner of the hot/cold water tank and extends along an adjacent wall of the hot/cold water tank. The other wall of the corner has a hole at the position corresponding to the port 906, so that water from the water manifold can be pumped into the hot/cold water tank. The curved tunnel shape of the water return guide 1102 can slow down the water flow for water returned from the water manifold 108. As can also be seen in FIG. 11B, the water return guide 1102 has an uncovered top surface, which flushes with the bottom surface of the major part of the hot/cold water tank. In other words, the water return guide 1102 is lower than the bottom of the hot/cold water tank. In some embodiments, the vertical bar 1004 is disposed at a potion at or proximate to the center of the curved portion of the water return guide 1102, as can be seen in FIG. 11B.

As further illustrated in FIGS. 11A-11B, a second corner of the hot/cold water further includes a valve 1104, which is a part of the drain solenoid valve 912 (shown in FIG. 9B) and is used for guiding water to be drained from the hot/cold water tank to the water reservoir tank. The position of the valve 1104 is aligned with the port 910, as can be seen from FIGS. 11A-11B. In some embodiments, the part holding the valve 1104 is in a half-tubing shape with the top surface flush with the bottom surface of the water return guide 1102, as can be seen further in FIG. 11B.

In some embodiments, the hot/cold water tank includes additional components not described above. For example, a rubber seal can be placed between the top part 902 and the bottom part 904 of the hot/cold water tank.

Referring now to FIGS. 11C-11D, various internal views of another example hot/cold water tank are provided, according to some embodiments. Different from the hall sensor in FIGS. 11A-11B that measures the distance from the bottom of the water tank, the hall sensor in FIG. 11D measures the change instead of the distance. For example, when the water level in the tank rises above a certain level (which can be considered as a threshold), the hall sensor begins to sense the change. Below the threshold level, the hall sensor may not sense any change of the water level. When the water level rises to the threshold level, the hall sensor begins to sense the change, and based on the change, it can be determined whether to continue to pump water from the main reservoir to the hot/cold water tank or not. In some embodiments, by just measuring the change but not the precise distance, the restrictive requirement of the hall sensor for precise distance measurement may not be necessary. In addition, by monitoring the change but not the precise distance of the water level in the hot/cold water tank, the control mechanism for controlling the water pump to pump water to the hot/cold water tank may be less complicated.

In some embodiments, since the hall sensor is intended to measure the change above the threshold but not the precise distance from the bottom of the water tank, the position of the magnetic float 1122 and the corresponding vertical bar 1004 is not limited to a specific location (e.g., limited to the water return guide 1102) as shown in FIGS. 11A-11B, but can be at any possible location, as can be seen from FIG. 11D.

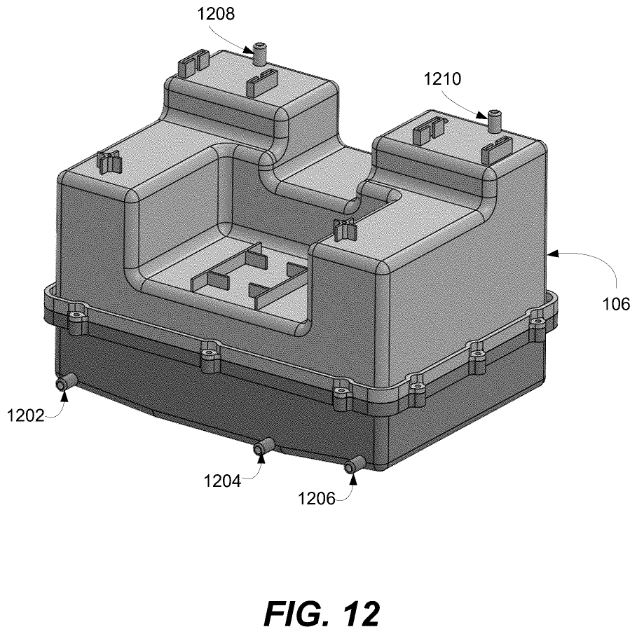

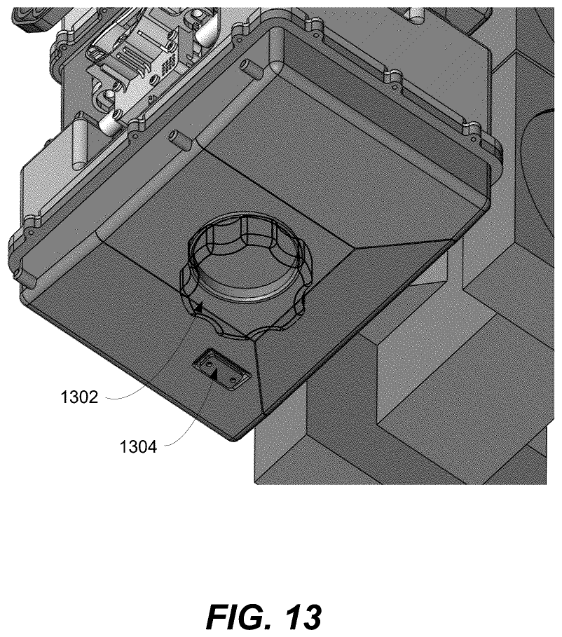

Referring now to FIGS. 12 and 13, the specific structure of the water reservoir tank is further illustrated, according to some embodiments. As illustrated in FIG. 12, the overall shape of the water reservoir tank can have a sofa chair shape with a missing middle part in the back portion of the “sofa chair,” which thus forms two concaved portions. In the front bottom part of the water reservoir tank 106, there are three ports 1202, 1204, and 1206. The first port 1202 is connected to the cold water tank and is used to receive water drained from the cold water tank. The second port 1204 is connected to the water manifold 108 and is used to provide water to the water manifold 108. The third port 1206 is connected to the hot water tank and is used to receive water drained from the hot water tank.

As further illustrated in FIG. 12, on the top left of the back portion of the water reservoir tank, there is also a port 1208 for receiving water overflown from the cold water tank. On the top right of the back portion of the tank, there is another port 1210 for receiving water overflown from the hot water tank. As can be seen from FIG. 12, the water reservoir tank also includes two separate parts that are secured through a fixing mechanism (e.g., a set of screws and a rubber seal).

As can be seen clearly from FIG. 2, the lower concaved portion can be configured to have a shape and/or size that fits the valve and manifold assembly and the upper concaved portion can be configured to have a shape and size that fits the air pump. Such configurations allow the overall structure of the contrast and compression therapy apparatus 100 to have a compact shape for better portability.

Referring to FIG. 13, the bottom structure of the water reservoir tank is further illustrated, according to some embodiments. As illustrated in the figure, the middle portion of the bottom includes a cap 1302, which can be screwed to remove, to allow a user to fill or empty water in the water reservoir tank. In some embodiments, the water reservoir tank further includes one or more hall sensors 1304 (only one hall sensor 1304 is shown in the figure). Although not shown, the inner part of the water reservoir tank can also include a magnetic float ring aligned along a vertical bar, similar to a hot/cold water tank described above. In some embodiments, the water reservoir tank includes additional components not described above, such as accessory components for attaching or fixing purposes.

In some embodiments, instead of measuring the exact water level based on the distance measurement using the hall sensors 1304, a different sensor that measures whether the water reservoir tank is empty or not is included in the tank. The sensor may work similarly to a sensor in a gas tank of a car. By using such a much simpler sensor, the corresponding control mechanism is much easier, since it does not require consistently monitoring the water level in the water reservoir tank. Only when the water tank becomes empty or close to empty, it triggers an alert signal to be generated (which can be displayed on a display screen), to alert the person in use to refill the water reservoir tank. In some embodiments, the whole contrast and compression therapy apparatus 100 is controlled to stop the treatment if the water reservoir tank is detected empty.

In some embodiments, water circulating within the contrast and compression therapy apparatus 100 may slowly lose due to evaporation or other possible reasons, which then causes the water in the water reservoir tank to become less and less until the tank becomes empty or close to empty (which the sensor disclosed herein can also measure like a sensor in a gas tank).

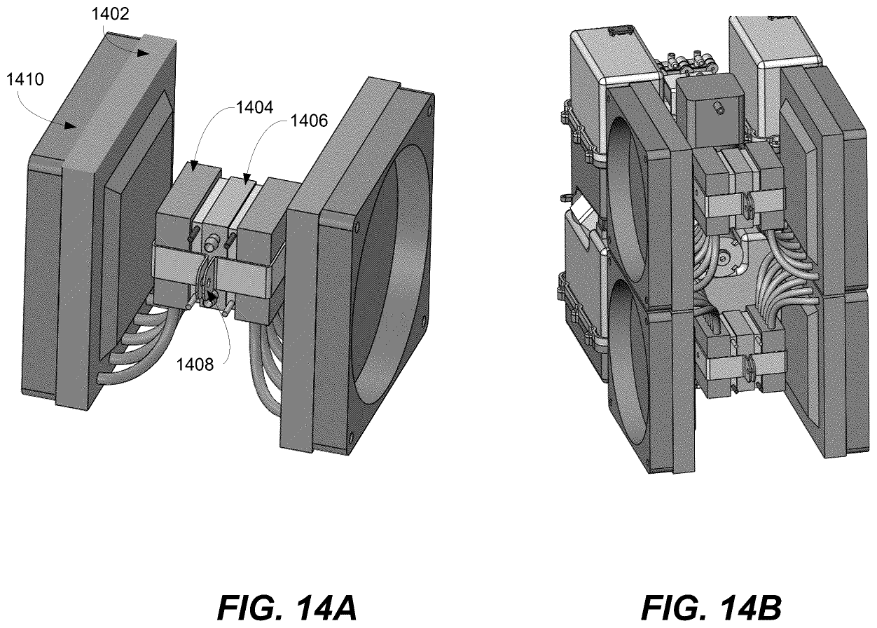

Referring now to FIGS. 14A-14B, the specific structure of a heat exchanger is further illustrated, according to some embodiments. As illustrated in FIG. 14A, the heat exchanger includes a pair of heatsinks 1402 with heat pipes, a pair of thermoelectric coolers (TECs) 1404, and a water block 1406 disposed between the two TECs. In some embodiments, there is an additional champing bracket 1408 that tightens the two TECs 1404 and the water block 1406 together.

A heatsink 1402 is a passive heat exchanger that transfers the heat generated by an electronic component (e.g., fins) to a fluid medium, such as water in the water block 1406. Although not illustrated, in some embodiments, the heatsink 1402 can include a plurality of fins horizontally extending from a vertically aligned base portion of the heatsink. In some embodiments, the plurality of fins can be formed through a skiving technique. In some embodiments, the plurality of fins can be referred to as skived fins. In some embodiments, in contrast to using extrusion, which is one way conventional heatsinks are formed, the entire heatsink 1402 can be formed using a skiving technique. In some embodiments, the heatsink 1402 can be referred to as a skived heatsink. For example, a metal work skiving process can be used to form the heatsink 1402 and/or the plurality of fins.

TEC 1404 uses the Peltier effect to create a heat flux at the junction of two different types of materials. A thermoelectric pump is a solid-state active pump that transfers heat from one side of the device to the other, with the consumption of electrical energy, depending on the direction of the current. It can be used either for heating or for cooling, and thus can also be used as a temperature controller that either heats or cools water passing through the water block.

In some embodiments, the heat exchanger is controlled by the PCB control unit, to allow the water to be either heated or cooled down to a target temperature. In some embodiments, the heat exchanger further includes a thermometer or sensor that can monitor the water temperature inside the water block 1406 so that the heat exchanger can be dynamically controlled to heat or cool water when water passes through the water block 1406.

In some embodiments, on the outer side of the heatsink 1402 away from the water block 1406, there is also a fan 1410 attached to the heatsink. The fan 1410 is mainly used to dissipate heat generated by the heat exchanger so that the contrast and compression therapy apparatus itself is not overheated. In some embodiments, there is more than one heat exchanger unit included in the contrast and compression therapy apparatus 100. For example, in the illustrated embodiment in FIG. 14B, there are two heat exchangers that are vertically aligned. Each of the two heat exchangers can operate independently or in a collaborative manner when heating or cooling the water for thermotherapy/cryotherapy.

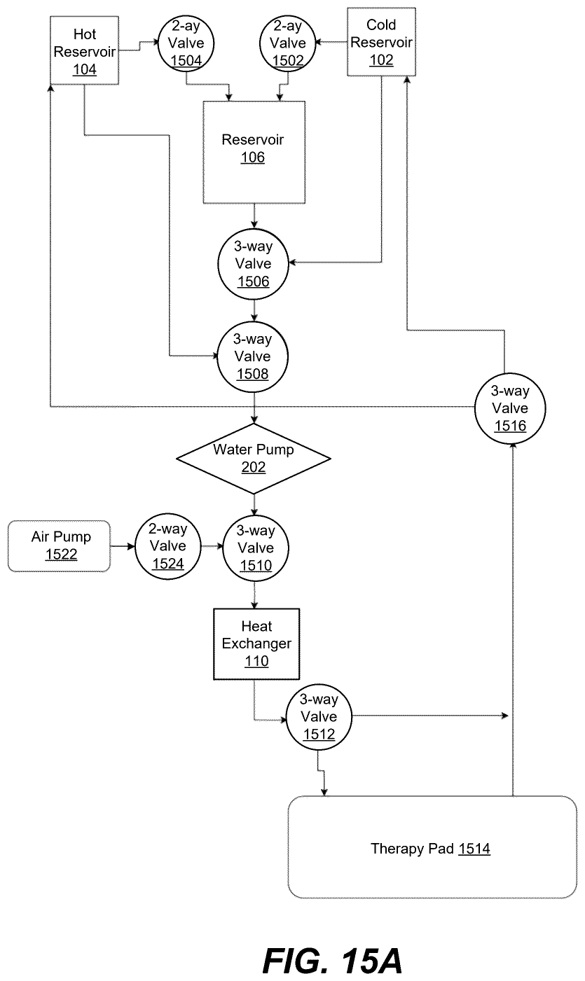

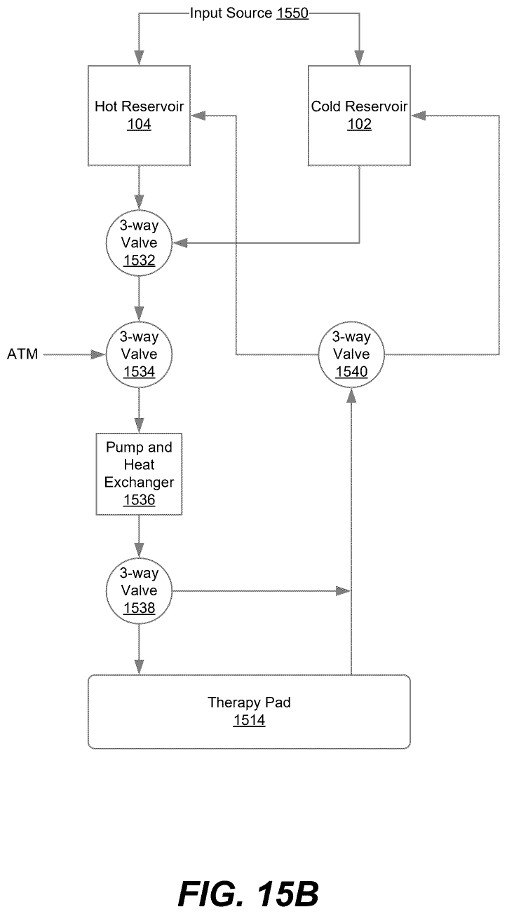

Referring now to FIG. 15A, a flow diagram for illustrating the water or air flow inside the contrast and compression therapy apparatus 100 is further illustrated, according to some embodiments. In the figure, the arrow indicates the flow direction of water or air in the device. For example, a 2-way valve 1502 controls water or air to flow from the cold water tank (or “cold reservoir”) 102 to the water reservoir tank (or simply “reservoir”) 106, a 2-way valve 1504 controls water or air to flow from the hot water tank (or “hot reservoir”) 104 to the water reservoir tank or reservoir 106, a 3-way valve 1506 controls water or air to flow from the reservoir 106 or from the cold reservoir 102 to a 3-way valve 1508, the 3-way valve 1508 controls water or air to flow from the 3-way valve 1506 or from the hot reservoir 104 to a water pump 202, a 3-way valve 1510 controls water or air to flow from the water pump 202 or control air to flow from the atmosphere (e.g., through air pump 1522 and 2-way valve 1524) to the heat exchanger 110, a 3-way valve 1512 controls water or air to flow from the heat exchanger 110 to either a therapy pad 1514 or directly to a 3-way valve 1516 without passing through the therapy pad 1514, and the 3-way valve 1516 controls water or air to flow from the therapy pad 1514 or the 3-way valve 1512 to the cold reservoir 102 or the hot reservoir 104. In some embodiments, by controlling the 2-way and 3-way valves to open/close and/or direct to proper channels, different circulation paths or loops can be formed through the illustrated configuration shown in FIG. 15A.

According to one embodiment, to build up cold water in the cold reservoir 102, a directional circulation path is formed following a route: reservoir 106>3-way valve 1506>3-way valve 1508>pump water 202>3-way valve 1510>heat exchanger 110>3-way valve 1512>3-way valve 1516>cold reservoir 102.

According to another embodiment, to build up hot water in the hot reservoir 104, a directional circulation path is formed following a route: reservoir 106>3-way valve 1506>3-way valve 1508>water pump 202>3-way valve 1510>heat exchanger 110>3-way valve 1512>3-way valve 1516>hot reservoir 104.

According to another embodiment, to implement a cryotherapy treatment, a directional circulation loop is formed following a route: cold reservoir 102>3-way valve 1506>3-way valve 1508>water pump 202>3-way valve 1510>heat exchanger 110>3-way valve 1512>therapy pad 1514>3-way valve 1516>cold reservoir 102.

According to another embodiment, to implement a thermotherapy treatment, a directional circulation loop is formed following a route: hot reservoir 104>3-way valve 1508>water pump 202>3-way valve 1510>heat exchanger 110>3-way valve 1512>therapy pad 1514>3-way valve 1516>hot reservoir 104.

According to another embodiment, to empty the cold reservoir 102 after a treatment, a directional circulation path is formed following a route: air pump 1522>2-way valve 1524>3-way valve 1510>heat exchanger 110>3-way valve 1512>therapy pad 1514 (just for partial of the emptying process so that the section between the 3-way valve 1512 and 3-way valve 1516 can also be emptied)>3-way valve 1516>cold reservoir 102>2-way valve 1502>reservoir 106.

According to another embodiment, to empty the hot reservoir 104 after a treatment, a directional circulation path is formed following a route: air pump 1522>2-way valve 1524>3-way valve 1510>heat exchanger 110>3-way valve 1512>therapy pad 1514 (just for partial of the emptying process so that the section between the 3-way valve 1512 and 3-way valve 1516 can also be emptied)>3-way valve 1516>hot reservoir 104>2-way valve 1504>reservoir 106.

In some embodiments, a proper path for flushing or emptying the tubings after hot water buildup in the hot reservoir 104 and another path for flushing or emptying the tubings after cold water buildup in the cold reservoir 102 can also be formed through proper control of the 2-way and/or 3-way valves. Additional paths or loops for pumping air into the therapy pad 1514 may also be formed through the proper control of the 2-way and/or 3-way valves, detail of which is not described herein.

In some embodiments, the water pump 202 is used to pump water during the treatments, while the air pump 1522 is used to flush the tubings after each session of treatment and/or empty the hot and cold reservoirs after each treatment if necessary. In some embodiments, the air pump 1522 may have a high efficiency than the water pump 202 for water repelling. For example, the air pump 1522 may have a water repelling rate of 6 liter/minute, while the water pump 202 may have a water repelling rate of 2 liter/minute. In some embodiments, by using the air pump 1522 to flush the tubings between the treatment sessions, the treatment efficiency can be improved, since much less time is required for flushing the tubings. In some embodiments, the air pump 1522 is also used to pump air into the therapy pad 1514 (e.g., bladder in the therapy pad). Accordingly, when the air pump 1522 is directed to flush the tubings or empty the hot/cold reservoirs, the air pump 1522 may pause pumping air into the therapy pad, and the bladder may remain in the deflated state under such circumstances (or may be in the inflated state if necessary). In some embodiments, there may be an additional air pump so that one air pump is responsible for repelling or pushing water inside the circulation loops, while the other air pump is used to pump air into the therapy pad 1514, which is not limited in the present disclosure.

As described earlier, under certain circumstances, the contrast and compression therapy apparatus 100 does not include a water reservoir tank. Accordingly, the water and air flow paths for such a configuration may also be different from what has been described in FIG. 15A. FIG. 15B illustrates a flow diagram for water and air flow for another contrast and compression therapy apparatus, according to some embodiments. The water manifold for controlling such water and air flow may refer to the illustrated embodiment in FIG. 6C.

At the top of the diagram, an input source 1550 supplies water that feeds into two separate reservoirs: a hot reservoir (104) and a cold reservoir (102). These reservoirs serve as the origin points for hot and cold water respectively. From the cold/hot reservoir 102/104, water flows downward through a first 3-way valve 1532, which controls whether water from the cold/hot reservoir 102/104 proceeds into the rest of the system or whether flow is diverted or halted. The next component in the sequence is a second 3-way valve 1534, which is strategically positioned to receive either water from the cold/hot reservoir 102/104 or air from the atmosphere (ATM). This valve determines whether water or air enters the system, allowing for purging or switching of media in the subsequent stages.

Downstream of valve 1534 is the pump and heat exchanger unit 1536. This unit is responsible for propelling the water through the system and adjusting its temperature as needed. The water is either maintained at its current thermal level or further cooled/heated depending on treatment requirements. From there, the water flows through a third 3-way valve 1538, which controls whether it proceeds into the therapy pad 1514 or is rerouted to another 3-way 1540 without necessarily passing through the therapy pad 1514. For example, under circumstances, hot/cold water reservoirs may not have desired temperatures and thus the hot/cold water may be pre-heated/cooled to the desired temperatures before the actual contrast and compression therapy or even during the actual therapy (e.g., between the alternating cryotherapy and thermotherapy sections where the hot/cold water does not pass through the therapy pad 1514 until the desired temperature is reached). Under such conditions, the water from the hot/cold reservoir may flow through the pump and heat exchanger 1536 to get heated or cooled there, and then directly flow back to the hot/cold reservoir through the 3-way valve 1540 without requiring to pass through the therapy pad 1514.

Similar to FIG. 15A, by controlling the 3-way valves to open/close and/or direct to proper channels, different circulation paths or loops can be formed through the illustrated configuration shown in FIG. 15B.

According to one embodiment, to cool the cold water in the cold reservoir 102 to a desired temperature through the pump and heat exchanger 1536, a directional circulation path is formed following a route: cold reservoir 102>3-way valve 1532>3-way valve 1534>pump and heat exchanger 1536 (where the cold water can be cooled to a desired temperature)>3-way valve 1538>3-way valve 1540>cold reservoir 102.

According to one embodiment, to heat the hot water in the hot reservoir 104 to a desired temperature through the pump and heat exchanger 1536, a directional circulation path is formed following a route: hot reservoir 104>3-way valve 1532>3-way valve 1534>pump and heat exchanger 1536 (where the hot water can be cooled to a desired temperature)>3-way valve 1538>3-way valve 1540>hot reservoir 104.

According to another embodiment, to implement a cryotherapy treatment, a directional circulation loop is formed following a route: cold reservoir 102>3-way valve 1532>3-way valve 1534>pump and heat exchanger 1536 (where the cold water can be cooled again to a desired temperature if necessary)>3-way valve 1538>therapy pad 1514>3-way valve 1540>cold reservoir 102.

According to another embodiment, to implement a thermotherapy treatment, a directional circulation loop is formed following a route: hot reservoir 104>3-way valve 1532>3-way valve 1534>pump and heat exchanger 1536 (where the hot water can be heated again to a desired temperature if necessary)>3-way valve 1538>therapy pad 1514>3-way valve 1540>hot reservoir 104.

According to another embodiment, to empty the cold reservoir 102 after a treatment, a directional air circulation path is formed following a route: atmosphere (ATM)>3-way valve 1534>pump and heat exchanger 1536>3-way valve 1538>therapy pad 1514 (just for partial of the emptying process so that the section between the 3-way valve 1528 and 3-way valve 1540 can also be emptied)>3-way valve 1540>cold reservoir 104>3-way valve 1532>3-way valve 1534.

According to another embodiment, to empty the hot reservoir 104 after a treatment, a directional air circulation path is formed following a route: atmosphere (ATM)>3-way valve 1534>pump and heat exchanger 1536>3-way valve 1538>therapy pad 1514 (just for partial of the emptying process so that the section between the 3-way valve 1528 and 3-way valve 1540 can also be emptied)>3-way valve 1540>hot reservoir 104>3-way valve 1532>3-way valve 1534.

From the above, it can be seen that the configuration in FIG. 15B allows for seamless alternation between hot and cold therapies while maintaining precise temperature control. The inclusion of atmospheric air routing through valve 1534 provides a means to purge the system, expel residual water between cycles, or enable compression therapy functions. The strategic placement and interaction of the valves and pump allow the system to operate without manual intervention, maintaining a closed-loop control over thermal therapy delivery.

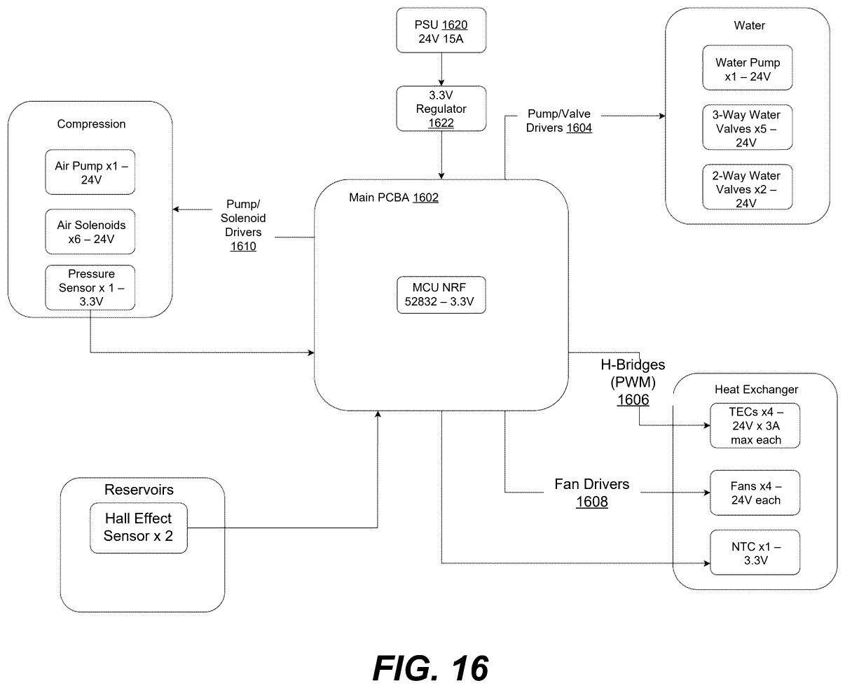

Referring to FIG. 16, a schematic diagram of an electronic system for the contrast and compression therapy apparatus 100 is illustrated, according to some embodiments. As illustrated in the figure, the PCB control unit includes a microcontroller unit 1602 and a set of drivers for driving different components included in the contrast and compression therapy apparatus 100. For example, the PCB control unit includes a set of pump/valve drivers 1604 for driving the water pump to turn on or off, for driving 2-way valves to open or close, and for driving 3-way valves to close or open in different directions. The PCB control unit also connects to a set of H-bridges 1606 to control the heat transfer of the TECs. In addition, the PCB control unit also includes a set of fan drivers for driving the fans to dissipate heat generated by the heat exchanger(s). In addition, the PCB control unit also collects signals obtained from the temperature sensors (such as negative temperature coefficient (NTC) sensors) included in the heat exchanger(s) to control heat or cold generated by the heat exchanger(s). Further, the PCB control unit can receive signals generated by the hall sensors from the reservoirs to determine the water volume in each reservoir. In some embodiments, the PCB control unit also includes a set of pump/solenoid drivers 1610 for turning on or off the air pump, for controlling the air solenoids, and for collecting information obtained from pressure sensors.

In some embodiments, the contrast and compression therapy apparatus also includes a power supply unit (PSU) 1620 for providing power to different components included in the contrast and compression therapy apparatus 100. These components include but are not limited to the air pump, water pump, various sensors, solenoids, PCB control unit, and heat exchanger(s), and heater (if there is one) in a therapy pad. In some embodiments, the power supply unit 1620 provides power to different components through the PCB control unit. In some embodiments, there is also a regulator 1622 coupled to the power supply unit 1620 and the PCB control unit 1602 to regulate the power supply to different components included in the contrast and compression therapy apparatus 100. This allows the contrast and compression therapy apparatus to operate in a desired manner.

### Computer Systems

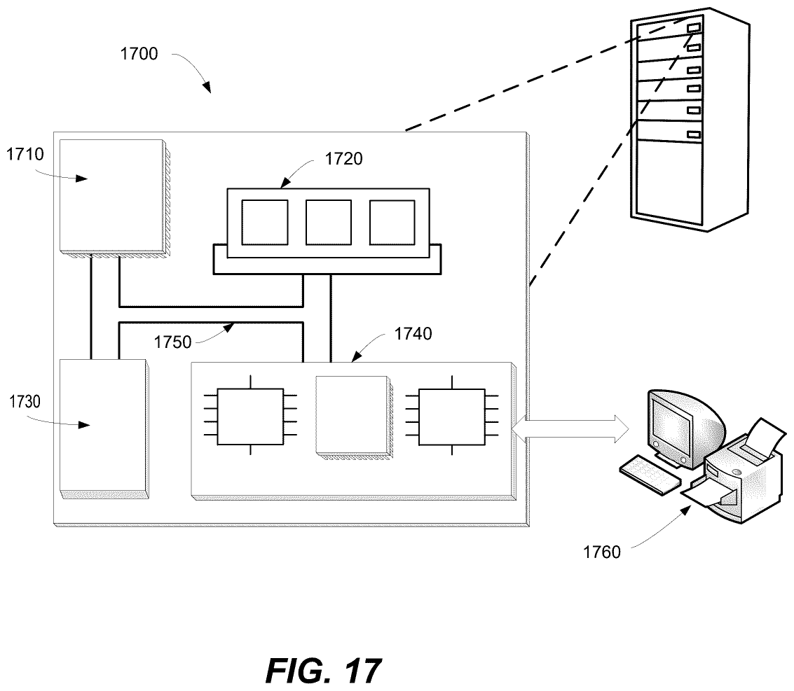

FIG. 17 is a block diagram of an example computer system 1700 that can be used in implementing the technology described in this document. General-purpose computers, network appliances, mobile devices, or other electronic systems can also include at least portions of the system 1700. The system 1700 includes a processor 1710, a memory 1720, a storage device 1730, and an input/output device 1740. Each of the components 1710, 1720, 1730, and 1740 can be interconnected, for example, using a system bus 1750. The processor 1710 is capable of processing instructions for execution within the system 1700. In some implementations, the processor 1710 is a single-threaded processor. In some implementations, the processor 1710 is a multi-threaded processor. The processor 1710 is capable of processing instructions stored in the memory 1720 or on the storage device 1730.

The memory 1720 stores information within the system 1700. In some implementations, the memory 1720 is a non-transitory computer-readable medium. In some implementations, the memory 1720 is a volatile memory unit. In some implementations, the memory 1720 is a non-volatile memory unit.

The storage device 1730 is capable of providing mass storage for the system 1700. In some implementations, the storage device 1730 is a non-transitory computer-readable medium. In various different implementations, the storage device 1730 can include, for example, a hard disk device, an optical disk device, a solid-date drive, a flash drive, or some other large-capacity storage device. For example, the storage device can store long-term data (e.g., database data, file system data, etc.). The input/output device 1740 provides input/output operations for the system 1700. In some implementations, the input/output device 1740 can include one or more network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device can include driver devices configured to receive input data and send output data to other input/output devices, e.g., keyboard, printer and display devices 1760. In some examples, mobile computing devices, mobile communication devices, and other devices can be used.

In some implementations, at least a portion of the approaches described above can be realized by instructions that upon execution cause one or more processing devices to carry out the processes and functions described above. Such instructions can include, for example, interpreted instructions such as script instructions, or executable code, or other instructions stored in a non-transitory computer readable medium. The storage device 1730 can be implemented in a distributed way over a network, for example as a server farm or a set of widely distributed servers, or can be implemented in a single computing device.

Although an example processing system has been described in FIG. 17, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” can encompass all kinds of apparatus, devices, and machines for processing data, including by way of example a programmable processor, a computer, or multiple processors or computers. A processing system can include special purpose logic circuitry, e.g., an FPGA (field programmable gate array) or an ASIC (application specific integrated circuit). A processing system can include, in addition to hardware, code that creates an execution environment for the computer program in question, e.g., code that constitutes processor firmware, a protocol stack, a database management system, an operating system, or a combination of one or more of them.

A computer program (which can also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing can be advantageous. Other steps or stages can be provided, or steps or stages can be eliminated, from the described processes. Accordingly, other implementations are within the scope of the following claims.

### Terminology

The phraseology and terminology used herein is for the purpose of description and should not be regarded as limiting.

Measurements, sizes, amounts, and the like can be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Furthermore, connections between components or systems within the figures are not intended to be limited to direct connections. Rather, data or signals between these components can be modified, re-formatted, or otherwise changed by intermediary components. Also, additional or fewer connections can be used. The terms “coupled,” “connected,” or “communicatively coupled” shall be understood to include direct connections, indirect connections through one or more intermediary devices, wireless connections, and so forth.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range can be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An apparatus for providing contrast and compression therapy, comprising:
a hot water tank, a cold water tank, a water pump, a heat exchanger, and a therapy pad, wherein the hot water tank and the cold water tank are configured to store water at respective target temperatures for alternating delivery to the therapy pad for alternating thermotherapy and cryotherapy;
a programmable control unit configured to automatically switch the alternating delivery of the stored water at the respective target temperatures to the therapy pad; and
an air pump configured to deliver pressurized air to the therapy pad to provide a compression therapy simultaneously with the thermotherapy or the cryotherapy.

2. The apparatus of claim 1, further comprising a water reservoir tank connected to both the hot water tank and the cold water tank via a set of two-way valves, wherein the water reservoir tank facilitates water reuse between therapy sessions.

3. The apparatus of claim 2, wherein the water reservoir tank is also fluidly connected to the heat exchanger and the water pump via one or more three-way valves, such that water from the water reservoir tank is pumped through the heat exchanger to condition hot and cold water to the respective target temperatures before being delivered to the therapy pad.

4. The apparatus of claim 1, wherein the apparatus operates without a water reservoir tank, and wherein hot and cold water are supplied directly from external sources to the hot water tank and the cold water tank, respectively.

5. The apparatus of claim 1, wherein the heat exchanger is disposed in a water flow path between the hot water tank or the cold water tank and the therapy pad and is configured to dynamically heat or cool water to the respective target temperatures during the contrast and compression therapy.

6. The apparatus of claim 5, wherein the heat exchanger includes a temperature sensor configured to monitor a temperature of water flowing to the therapy pad and adjust the heating or cooling to maintain water at the respective target temperatures.

7. The apparatus of claim 1, wherein the therapy pad comprises at least two independent layers, including a fluid circulation layer for thermal therapy and an inflatable bladder for compression therapy.

8. The apparatus of claim 1, wherein the water pump and the air pump are physically integrated into a single unit configured to operate in alternating or parallel modes.

9. The apparatus of claim 1, wherein the hot water tank, the cold water tank, and optional water reservoir tank each include one or more hall effect sensors configured to monitor fluid levels within each tank.

10. The apparatus of claim 1, further comprising a water manifold configured to direct flow between multiple paths via a set of 2-way and 3-way valves, thereby forming closed-loop circulation paths for heating and cooling cycles.

11. The apparatus of claim 10, wherein the water manifold is connected to the therapy pad, the water pump, and the air pump, and is configured to switch a direction of flow between thermotherapy and cryotherapy cycles without requiring manual intervention.

12. The apparatus of claim 1, wherein the control unit is further configured to regulate a compression cycle timing.

13. The apparatus of claim 1, further comprising a detachable pad connector configured to interface with different therapy pads adapted for various anatomical regions.

14. The apparatus of claim 1, wherein the apparatus is housed within a compact, portable casing and further includes a rechargeable power supply for self-contained operation.

15. The apparatus of claim 14, wherein the rechargeable power supply is configured to provide power to the water pump, the air pump, the control unit, sensors, valves, and the heat exchanger without requiring an external power source during operation.

16. The apparatus of claim 1, wherein dynamic temperature regulation is enabled through real-time feedback from the heat exchanger and the control unit.

17. A system for delivering alternating thermotherapy and cryotherapy, comprising: a hot water tank, a cold water tank, a therapy pad, a heat exchanger, a plurality of valves, and a control unit, wherein the control unit is configured to:
activate a water pump to direct water from the hot water tank through the heat exchanger and into the therapy pad for a first treatment cycle;
subsequently activate one or more valves to route cold water from the cold water tank through the heat exchanger and into the therapy pad for a second treatment cycle; and
repeat alternating cycles based on a user-defined or preset timing schedule for alternating thermotherapy and cryotherapy.

18. The system of claim 17, wherein the control unit further directs an air pump to supply pressurized air to a compressive layer of the therapy pad concurrently with delivery of hot or cold water, thereby applying compression in synchrony with the alternating thermotherapy and cryotherapy.

19. The system of claim 17, wherein water temperature is monitored by one or more sensors positioned within or adjacent to the heat exchanger, and wherein the control unit adjusts heating or cooling output to maintain respective target temperatures throughout the first treatment cycle or the second treatment cycle.

20. The system of claim 17, wherein the apparatus operates in a reservoir-free configuration, and the control unit is further configured to flush fluid lines with air between treatment cycles to prevent mixing of residual thermal media, using solenoid-actuated valves to direct airflow through same circulation paths used for water delivery.
